(19) United States
(12) Patent Application Publication
(10) Pub. No.: US 2018/0126094 A1
(43) Pub. Date: May 10, 2018

(54) METHODS AND APPARATUS FOR SYRINGE ADAPTER

(71) Applicant: Alan Reid, Keene, NH (US)

(72) Inventor: Alan Reid, Keene, NH (US)

(21) Appl. No.: 15/804,184

(22) Filed: Nov. 6, 2017

Related U.S. Application Data
(60) Provisional application No. 62/418,423, filed on Nov. 7, 2016.

(51) Int. Cl. A61M 5/34; A61J 1/20; A61M 5/145; A61J 15/00

(52) U.S. Cl. CPC A61M 5/345; A61J 1/2096; A61M 2005/3131; A61J 15/0026; A61M 5/1458

(57) ABSTRACT

An assembly and syringe adapter is provided. In one embodiment, an assembly includes a syringe and a syringe adapter. The syringe has a fluid reservoir and a tip that is in communication with the fluid reservoir. Additionally, the adapter has first and second ends, the first end including a cavity configured to receive the syringe tip, and a mating mechanism to permanently engage the syringe tip when the syringe tip is received in the cavity and the adapter is rotated in a first direction relative to the syringe. The adapter may not alter a volume of the syringe to maintain accuracy of volume indicia on the syringe.

### CROSS REFERENCE TO RELATED APPLICATIONS

The present application claims the benefit of U.S. Provisional Patent Application No. 62/418,423, filed on Nov. 7, 2016, which is incorporated herein by reference.

### BACKGROUND

As is known in the art, a variety of syringes are used for medical applications. For example, an enteral syringe can be filled with nutritional material that can be ingested by a patient via a feeding tube. Another type of syringe having a threaded end to engage with a complementary end is used to provide solution intravenously. It is further known that an IV (intravenous) syringe can be filled with a substance, such as breast milk for an infant, and then misused as an IV syringe. This can lead to disastrous results including death due to substances being injected directly into a patient's vein.

### SUMMARY

The present disclosure provides methods and apparatus to adapt a syringe for enteral applications where the adapted syringe cannot be used for IV applications after being adapted. Additionally, the present disclosure provides methods and apparatus for adapting or converting a fluid dispensing output of an enteral or non-luer (i.e., non-IV) syringe for use with a variety of accessories and/or for use in a variety of applications.

In exemplary embodiments, an assembly includes an adapter and a cap, where the cap is used to rotate the adapter onto threads of the syringe for permanent engagement to the syringe to enable enteral feeding applications of the syringe. Rotation of the cap in the opposite direction removes the cap from the adapter to allow fluid egress from the syringe.

In one aspect of the disclosure, an assembly comprises an adapter having a first end and a second end and a cap engagement mechanism, the first end having a mating mechanism to engage a syringe, and a cap having an adapter engagement mechanism to engage the cap engagement mechanism of the adapter, wherein the adapter mating mechanism is configured to engage the syringe by rotation of the adapter in a first direction and to resist rotation of the adapter in a second direction opposite the first direction.

The assembly can further include one or more of the following features: the cap engagement mechanism and the adapter engagement mechanism are configured to rotate the adapter in the first direction by rotation of the cap in the first direction, rotation of the cap in the second direction disengages the cap from the adapter, the cap and adapter form a seal that is broken by rotation of the cap in the second direction, the first end includes a tip configured for enteral feeding, the cap engagement mechanism includes at least one tooth, the adapter engagement mechanism includes at least one tooth to engage the at least one tooth of the cap engagement mechanism, the mating mechanism includes one or more barbs to provide unidirectional rotation of the adapter when engaging the syringe, and the one or more barb extends from at least a partial thread.

In another aspect of the disclosure, a device comprises an adapter having a first end and a second end, the first end having a mating mechanism to engage a syringe, and wherein the adapter mating mechanism is configured to engage the syringe by rotation of the adapter in a first direction and to resist rotation of the adapter in a second direction opposite the first direction.

In a further aspect of the disclosure, a method comprises providing an adapter having a first end and a second end and a cap engagement mechanism, the first end having a mating mechanism to engage a syringe, and providing a cap having an adapter engagement mechanism to engage the cap engagement mechanism of the adapter, wherein the adapter mating mechanism is configured to engage the syringe by rotation of the adapter in a first direction and to resist rotation of the adapter in a second direction opposite the first direction.

In another aspect, a method comprises: securing an adapter to a fluid-dispensing end of a syringe such that the adapter is permanently secured to the syringe, which has indicia corresponding to fluid volume in the syringe, wherein the adapter does not modify the fluid volume of the syringe such that accuracy of the indicia is maintained after the adapter is secured to the syringe.

A method can further include one or more of the following features: the syringe comprises an enteral feeding syringe, the syringe comprises a non-luer type syringe, the adapter comprises a mating mechanism to secure the adapter to the syringe, the mating mechanism comprises self-threading threads, the mating mechanism is configured for unidirectional rotation, the mating mechanism comprises an adhesive, coupling a first accessory to the adapter for receiving fluid from the syringe, the first accessory comprises tubing, and/or coupling a second accessory to the first assembly for dispensing fluid to a mouth of a patient, providing an ISO-80369-3 compliant enteral feeding system.

In a further aspect, an assembly comprises: a syringe having a fluid reservoir and a tip that is in communication with the fluid reservoir; and an adapter having first and second ends, the first end including a cavity dimensioned to receive the syringe tip, and a mating mechanism to permanently engage the syringe tip when the syringe tip is received in the cavity, wherein the syringe includes indicia corresponding to fluid volume in the syringe, wherein the adapter does not modify the fluid volume of the syringe such that accuracy of the indicia is maintained after the adapter is secured to the syringe.

An assembly can further include one or more of the following features: the syringe comprises an enteral feeding syringe, the syringe comprises a non-luer type syringe, the adapter comprises a mating mechanism to secure the adapter to the syringe, the mating mechanism comprises self-threading threads, the mating mechanism is configured for unidirectional rotation, the mating mechanism comprises an adhesive, coupling a first accessory to the adapter for receiving fluid from the syringe, the first accessory comprises tubing, and/or coupling a second accessory to the first assembly for dispensing fluid to a mouth of a patient, providing an ISO-80369-3 compliant enteral feeding system.

### BRIEF DESCRIPTION OF THE DRAWINGS

The features and other details of the concepts, systems, and techniques sought to be protected herein will now be more particularly described. It will be understood that any specific embodiments described herein are shown by way of illustration and not as limitations of the disclosure and the concepts described herein. Features of the subject matter described herein can be employed in various embodiments without departing from the scope of the concepts sought to be protected. Embodiments of the present disclosure and associated advantages may be best understood by referring to the drawings, where like numerals are used for like and corresponding parts throughout the various views. It should, of course, be appreciated that elements shown in the figures are not necessarily drawn to scale. For example, the dimensions of some elements may be exaggerated relative to other elements for clarity.

FIG. 1 is a perspective schematic depiction of a syringe adapter in accordance with exemplary embodiments of the disclosure;

FIG. 2 is a perspective schematic depiction of a syringe adapter cap in accordance with exemplary embodiments of the disclosure;

FIG. 3 is a perspective schematic depiction of a syringe adapter/cap assembly in accordance with exemplary embodiments of the disclosure;

FIG. 3A is a cross-sectional view of a syringe cap/adapter assembly in accordance with exemplary embodiments of the disclosure;

FIG. 3B is a side view of a syringe cap/adapter assembly in accordance with exemplary embodiments of the disclosure;

FIG. 3C is a partial cutaway view of a cap for a syringe adapter in accordance with exemplary embodiments of the disclosure;

FIG. 3D is a perspective partial cutaway view of a cap for a syringe adapter in accordance with exemplary embodiments of the disclosure;

FIG. 4 is a schematic depiction of a prior art syringe to which a syringe adapter can be attached in accordance with exemplary embodiments of the disclosure;

FIG. 5 is a flow diagram showing an exemplary sequence of steps for a syringe adapter in accordance with exemplary embodiments of the disclosure;

FIG. 6 is a side view of an example syringe adapter according to the disclosure;

FIG. 6A is a top plan view of a first portion of the example syringe adapter of FIG. 6;

FIG. 6B is a bottom plan view of a second portion of the example syringe adapter of FIG. 6;

FIG. 6C is another side view of the example syringe adapter of FIG. 6;

FIG. 6D is a cross-sectional view of the example syringe adapter of FIG. 6 taken across lines A-A shown in FIG. 6C;

FIG. 6E is a magnified view of a portion of the example syringe adapter of FIG. 6;

FIG. 7 is a side view of an example syringe adapter according to the disclosure and a prior art syringe to which the syringe adapter can be attached;

FIG. 7A is a perspective view of the example syringe adapter and the prior art syringe of FIG. 7;

FIG. 8 is a perspective view of the example syringe adapter of FIG. 7 attached to the prior art syringe;

FIG. 8A is another perspective view of the example syringe adapter of FIG. 7 attached to a prior art syringe;

FIG. 8B is a perspective view of the example syringe adapter of FIG. 7 attached to a prior art syringe, and of example accessories that may be coupled to the syringe adapter; and

FIG. 9 is a perspective view of the example syringe adapter of FIG. 7 attached to another prior art syringe.

### DETAILED DESCRIPTION

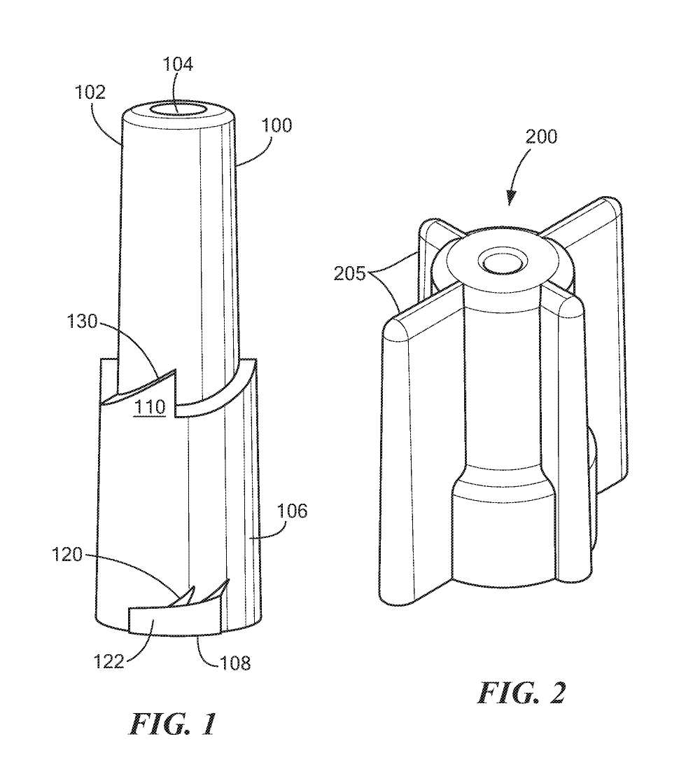

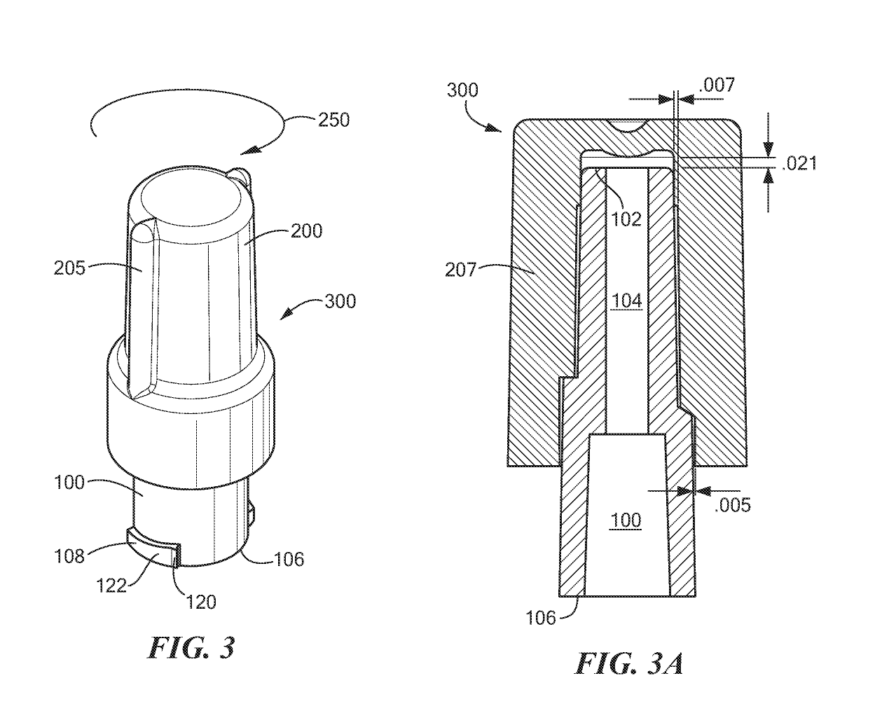

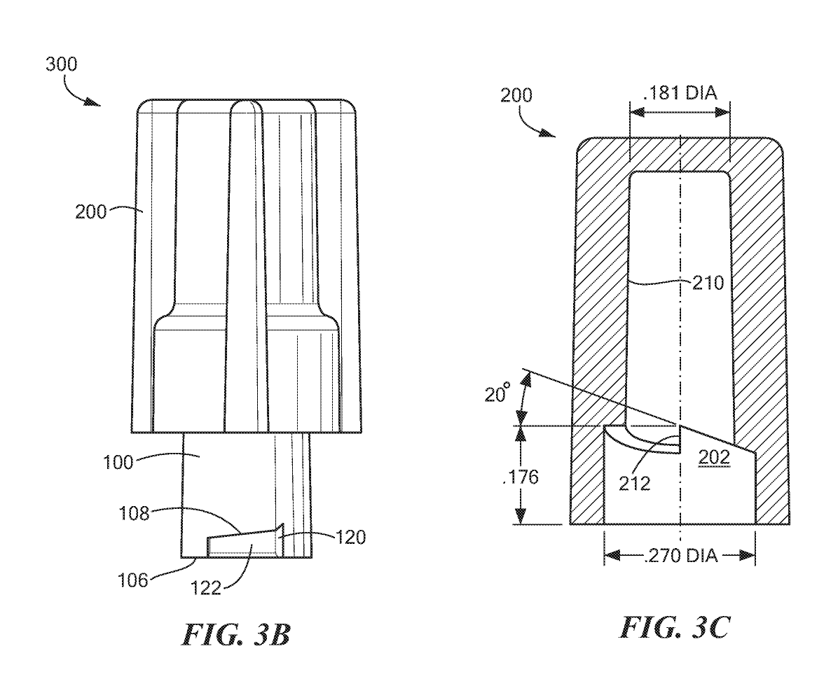

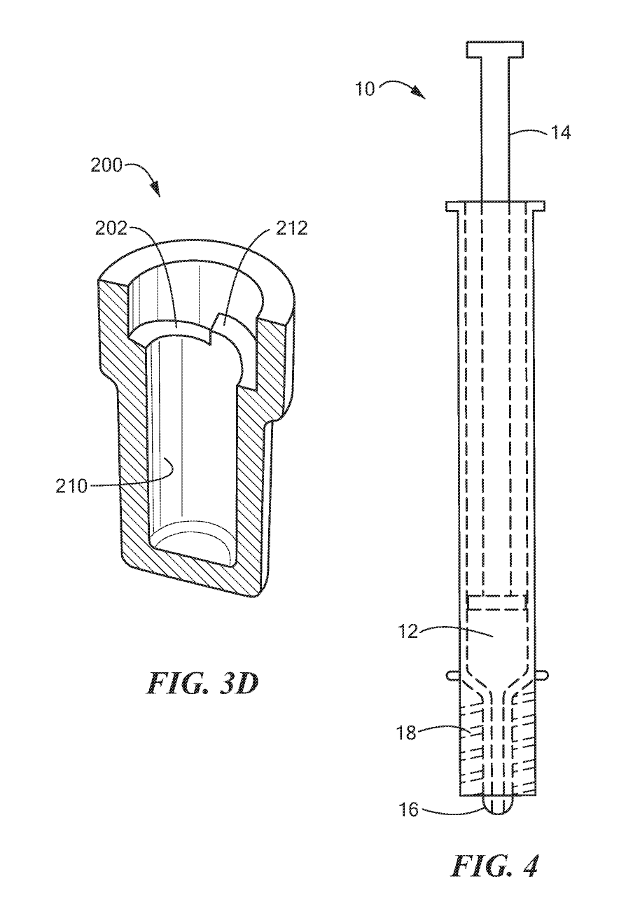

FIG. 1 shows an exemplary embodiment of a syringe adapter 100 and FIG. 2 shows an exemplary embodiment of a cap 200 that is matable with the syringe adapter 100. The adapter/cap assembly 300 of FIG. 3 can be coupled to a syringe 10, shown in FIG. 4, to safely adapt the syringe for enteral feeding applications, as described in detail below.

The adapter/cap assembly 300 can be secured to the syringe 10 of FIG. 4. The syringe 10 can be a conventional IV syringe having a fluid reservoir 12 from which liquid is forced out by depressing a plunger 14. Fluid exits the syringe 10 via a tip 16 in communication with the fluid reservoir 12. A distal end of the syringe includes internal threads 18 to receive a device having complementary threads.

As described in detail below, the adapter/cap assembly 300 can be threadably engaged with threads 18 on the syringe to enable the syringe to be safely used for enteral feeding since the adapter 100 (FIG. 1) cannot be non-destructively removed from the syringe 10. Since the adapter 100 cannot be removed, after enteral feeding applications, the syringe cannot be used for IV applications. In an exemplary embodiment, the adapter 100 and/or the cap 200 can be colored orange, at least in part, to indicated enteral feeding equipment.

Referring to FIGS. 1-4, including FIGS. 3A-3D, the adapter 100 includes a first end 102 having an opening 104 and a second end 106 with a mating mechanism 108 to engage threads 18 of the syringe 10 in a unidirectional attachment. More particularly, the mating mechanism 108 is configured to engage the threads 18 as the adapter 100 is turned in a first direction until full engagement is achieved. Once attached, however, the mating mechanism 108 of the adapter prevents the adapter 100 from being turned in the opposite direction. That is, the adapter 100 cannot be unthreaded from the syringe 10.

In an exemplary embodiment, the mating mechanism 108 includes one or more barbs 120 extending from an end of a partial thread 122 extending from the body of the adapter 100. The barb 120 and thread 122 engage the threads 18 on the syringe. The barb(s) 120 resist movement in the opposite direction so that the adapter 100 remains secured to the syringe 10.

It is understood that the mating mechanism can be provided in a variety of alternative suitable configurations that provide the desired one-way, permanent engagement of the adapter 100 to the syringe 10.

In an exemplary embodiment, the adapter 100 is engaged with the syringe 10 by means of a cap 200 coupled to the adapter. The adapter 100 includes a cap engagement mechanism 110 to engage the cap. The cap 200 includes a complementary adapter engagement mechanism 202 (FIG. 3C, 3D) to engage the cap engagement mechanism 110 so that rotation of the cap in a first direction 250 causes the adapter mating mechanism 108 to engage the threaded end 12 of the syringe. Rotation of the cap 200 in the opposite direction causes the cap to be dislodged from the adapter 100. With this arrangement, the cap 100 provides a uni-directional ratchet. Wings 205 on the cap 200, can facilitate rotation of the cap and assembly.

In one embodiment, the cap engagement mechanism 110 includes teeth 130 formed about the adapter 100. The teeth 130 transition a diameter for the tapered first end 102 to a diameter for the tapered second end 106, where the first end is smaller in diameter than the second end.

In an exemplary embodiment, the cap 200 includes an interior surface 210 having the adapter engagement mechanism 202 in the form of teeth 212 to engage the teeth 130 of the cap engagement mechanism of the adapter. While the cap 200 is coupled to the adapter 100, rotation of the cap 200 in a first direction 250 causes the adapter 100 to rotate and thereby be threaded onto the syringe. However, rotation of the cap 200 in the opposite direction causes the cap to disengage from the adapter 100 as the teeth of the 130 of the adapter cap engagement mechanism push off the teeth 212 of the cap adapter engagement mechanism 202.

It is understood that the cap and adapter engagement mechanisms can be provided in configurations other than complementary teeth. Other suitable one-way ratchet mechanisms will be readily apparent to one of ordinary skill in the art without departing from the present disclosure.

In one embodiment, the cap engagement mechanism 110 and the adapter engagement mechanism 202 provide a seal. In one embodiment, the seal is provided by an interference fit between the cap 200 and the adapter 100 proximate an end 102 of the adapter. Once the cap 200 is removed, fluid can pass through the adapter 200 to enable enteral feeding applications for example.

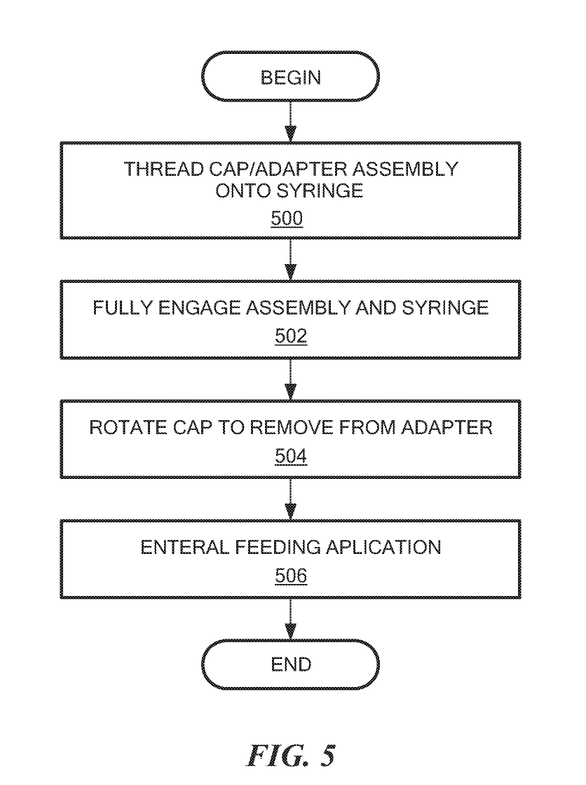

FIG. 5 shows an exemplary sequence of steps for providing a syringe adapter in accordance with exemplary embodiments of the disclosure. In step 500, a cap/adapter assembly is rotated in a first direction to thread the assembly onto a syringe. The cap includes an adapter engagement mechanism for coupling to a cap engagement mechanism of the adapter for enabling one-directional rotation of the cap/adapter assembly. In step 502, a mating mechanism of the adapter engages the threads of the syringe and the adapter is fully threaded onto the syringe. The mating mechanism resists rotation of the adapter in the opposite direction to prevent the adapter from being unthreaded from the syringe.

In step 504, the cap is rotated in the opposite direction so that the cap is pushed off the adapter. In an exemplary embodiment, this rotation breaks a seal between the cap and adapter to enable fluid to exit the syringe for allowing enteral feeding material to be ingested by a patient. In step 506, the adapted syringe is ready for enteral feeding applications.

In general, the adapter 100 and cap 200 have dimensions sized to meet the needs of a particular application. In the exemplary embodiments contained here, the adapter 100 and cap 200 are shown and described for attachment to a standard IV syringe. FIG. 3C shows certain exemplary dimensions, in inches, for the cap 200, and by extension, the adapter 100. While the described drawings may not be to scale, the drawings convey a general dimensional relationship between the components.

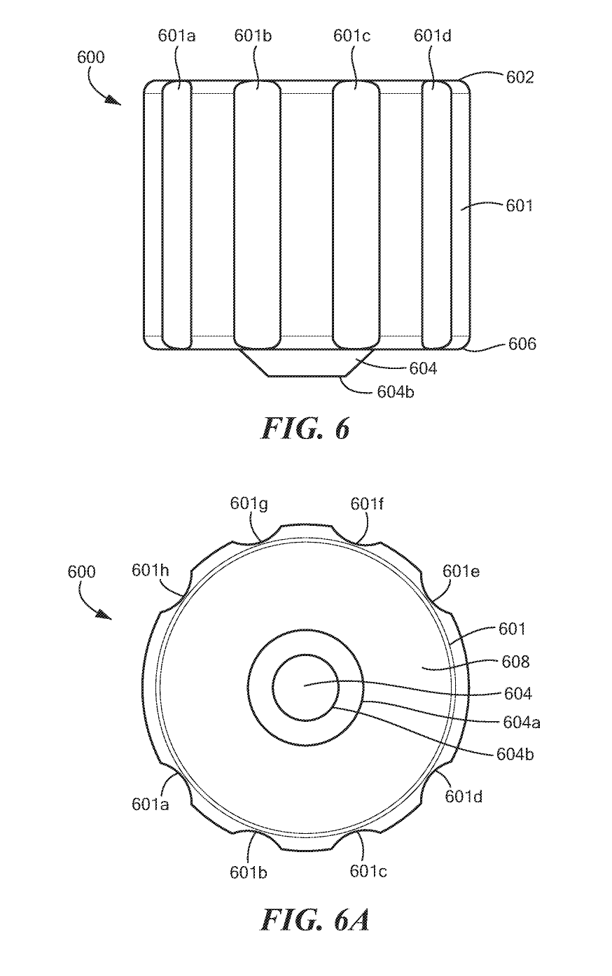

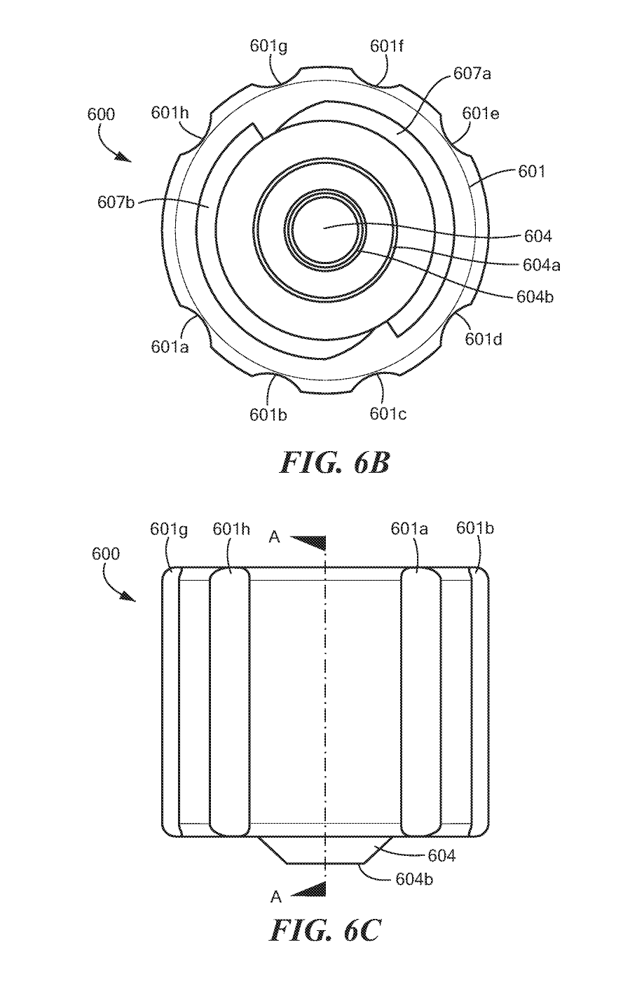

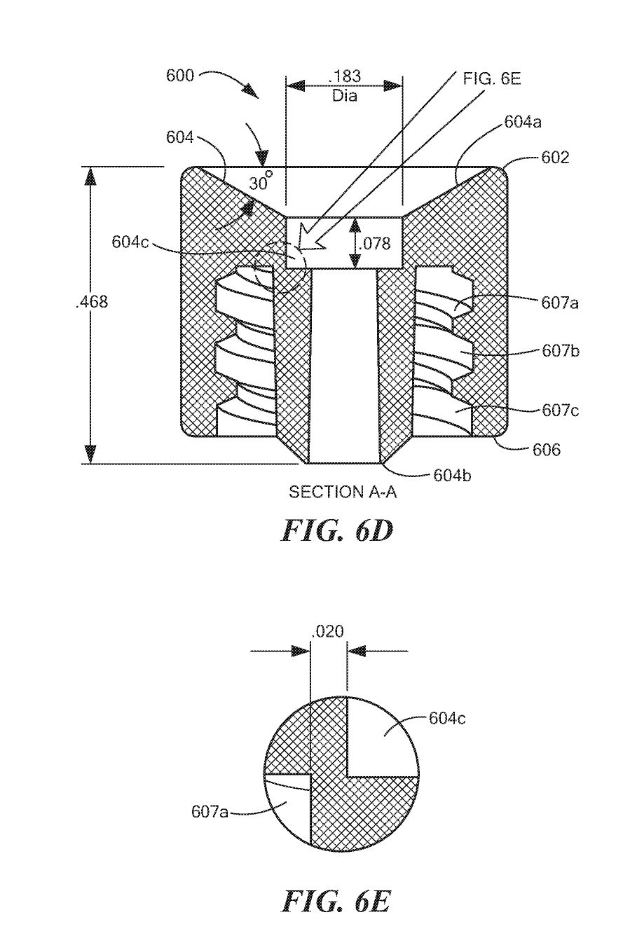

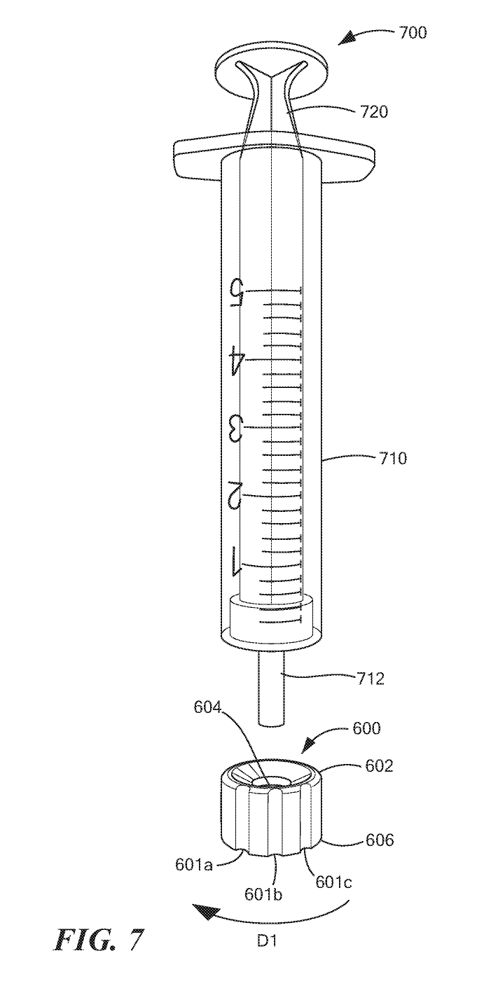

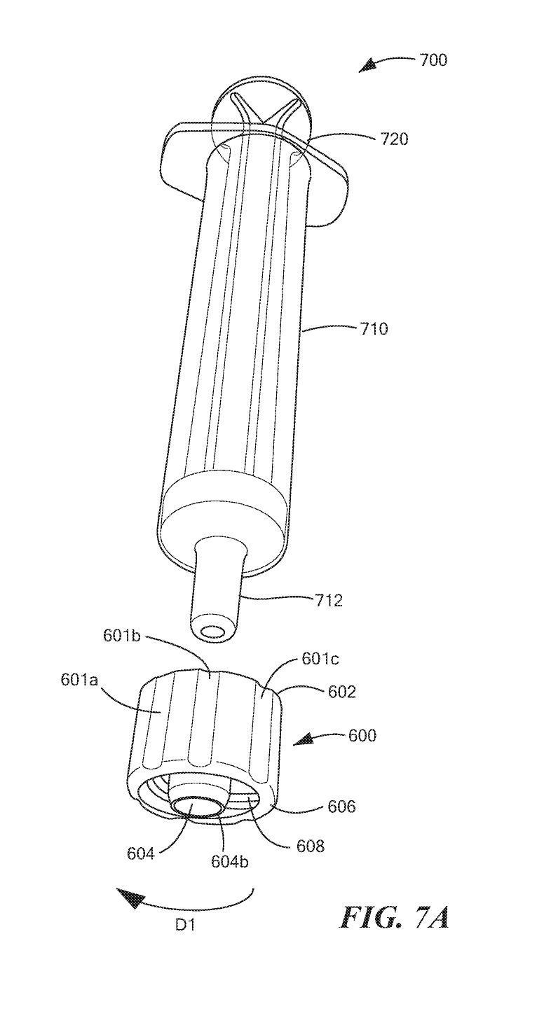

Referring to FIGS. 6-9, an example syringe adapter 600 according to another embodiment of the disclosure is shown. The syringe adapter 600 can be permanently attached or affixed to the syringe 700 of FIG. 7 to adapt or convert a fluid dispensing output of the syringe 700, as described in detail below. The syringe 700 can be a conventional enteral or non-luer (i.e., non-IV) syringe having a fluid reservoir 710 from which fluid (e.g., an enteral feeding medium) is forced out by depressing a plunger 720. Fluid exits the syringe 700 via a fluid dispensing output (here, a nozzle or tip 712) which is in communication with the fluid reservoir 710. In particular, the syringe tip 712, which may be a conventional enteral or non-luer tip, is in communication with the fluid reservoir 710 such that once the plunger 720 is depressed, fluid can pass through the fluid reservoir 710 to the syringe tip 712 to enable enteral feeding or other suitable applications. Generally, the syringe tip 712 is capable of being coupled to one or more accessories (e.g., feeding tubes) which are compatible with the syringe 700 and the syringe tip 712.

The syringe adapter 600 for attachment to the syringe 700 includes a first end 602 having a cavity 604 and a second, opposing end 606 having a cavity 608. The cavity 604 is dimensioned to receive the syringe tip 712 and includes a mating mechanism for engaging with the syringe tip 712. More particularly, the cavity 604 includes a first, end 604a for receiving the syringe tip 712 and a second, opposing end 604b. The second end 604b is proximate to the second end 606 of the syringe adapter 600 in the illustrated embodiment. Fluid stored in the fluid reservoir 710 of the syringe 700 may, for example, pass through the syringe tip 712 and exit from the second end 604b of the cavity 604 upon depression of the syringe plunger 720.

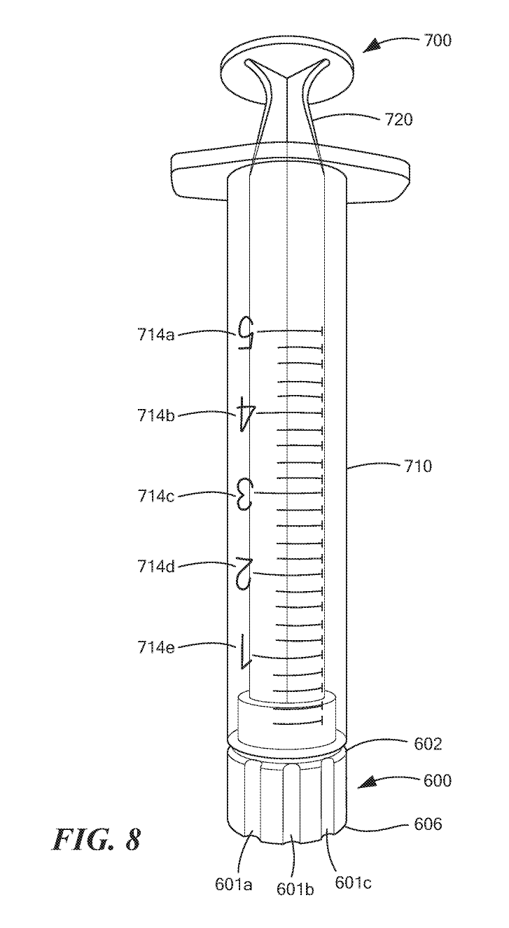

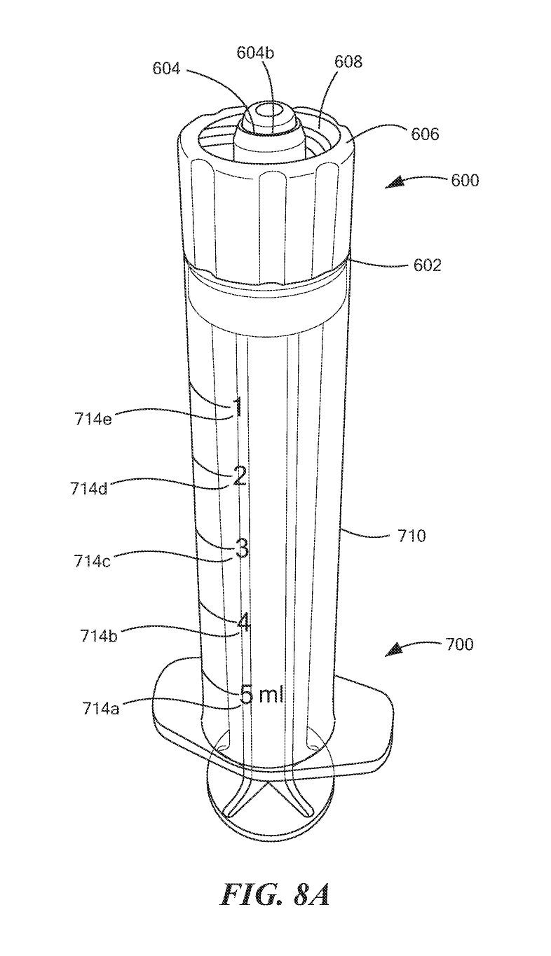

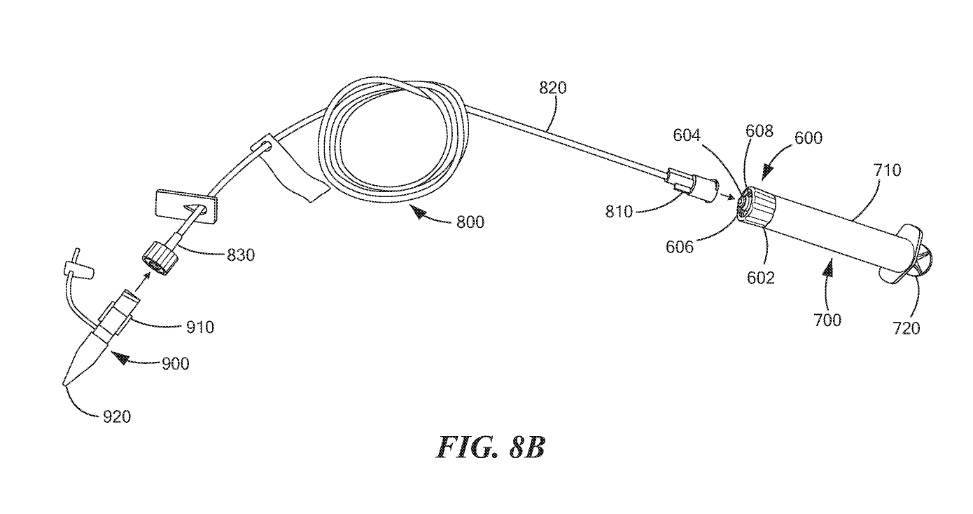

The mating mechanism of the syringe adapter 600, which comprises a plurality of threads 607a, 607b, etc. in the illustrated embodiment, as shown in FIGS. 6B and 6E, for example, is configured to engage the adaptor 600 with the syringe tip 712 when the syringe tip 712 is received in the cavity 604 and the adapter 600 is rotated in a first direction relative to the syringe 700, as shown in FIGS. 7-8A. Once the adapter 600 is attached to the syringe tip 712 via the mating mechanism, the mating mechanism prevents the adapter 600 from being turned in a direction that is opposite to the first direction D1. That is, the adapter 600 cannot be unthreaded from the syringe 700 (i.e., the adapter 600 is permanently attached to the syringe 700) once the mating mechanism is engaged with the syringe tip 712, as shown in FIGS. 8-8B. In some embodiments, the mating mechanism can also prevent the adapter 600 from being pulled away from, or generally removed from, the syringe tip 712.

The threads (e.g., helical threads) 607a, 607b, 607c, which extend a selected length of the cavity 604, as shown in FIG. 6D, self-thread onto the syringe tip 712 in the illustrated embodiment. In such self-threading embodiment, the adapter 600 should generally be formed from plastics (e.g., Polyvinyl chloride (PVC)) or other polymers of sufficient hardness to enable the threads 607a, 607b, 607c of the adapter 600 to self-thread onto the syringe tip 712 during rotation of the adapter 600 in a first direction D1 relative to the syringe 700, as shown in FIGS. 7 and 7A. It is understood that materials of the adapter 600 and, more particularly, materials of the mating mechanism of the adapter 600, may be selected based on materials from which the syringe tip 712 is provided (e.g., polypropylene). Additionally, it is understood that properties of the mating mechanism (e.g., a number, pitch, depth, etc. of the threads 607a, 607b, 607c) may be selected based upon dimensions or other properties (e.g., materials) of the syringe tip 712.

As discussed above, the mating mechanism prevents the adapter 600 from being turned in a direction that is opposite to the first direction D1 once the adapter 600 is attached to the syringe tip 712. In some embodiments, the threads 607a, 607b, 607c may sufficiently penetrate a surface of the syringe tip 712 to prevent reverse rotation of the adapter 600 relative to the syringe 700. In other embodiments, the mating mechanism (and the adapter 600, in general) is permanently attached or affixed to the syringe tip 712 additionally or alternatively using glue, an epoxy or another adhesive. Additionally, in other embodiments, the mating mechanism is permanently attached to the syringe tip 712 using one or more screws, by welding the mating mechanism (and/or other portions of the adapter 600) to the syringe tip 712, using a clamp and/or using one or more other permanent fastening or coupling means as known those of ordinary skill in the art. In other words, the adapter 600 is permanently attached to the syringe tip 712 once the mating mechanism is engaged with the syringe tip 712, as shown in FIGS. 8-8B. In some embodiments, a gap 604c may exist between the threads 607a, 607b, 607c of the mating mechanism and one or more portions of the cavity 604, as shown in FIG. 6E.

It is understood that the mating mechanism can be provided in a variety of alternative suitable configurations that provide the desired one-way, permanent attachment of the adapter 600 to the syringe 700. Other suitable mating mechanisms will be readily apparent to one of ordinary skill in the art without departing from the present disclosure. It is also understood that while the mating mechanism discussed above is described as having threads which self-thread onto syringe tip 712 of syringe 700, in embodiments in which the syringe tip 712 comprises threads, the threads of the mating mechanism may mate with corresponding threads of the syringe tip 712 when the adapter 600 is rotated in the first direction D1 relative to the syringe 700.

Referring to FIG. 6, an outer surface 601 of the adapter 600 may include a gripping means to facilitate rotation of the adapter 600 and the mating mechanism relative to the syringe tip 712 and, therefore, attachment of the adapter 600 to the syringe 700. For example, the gripping means may comprise a plurality of vertically extending ribs 601a, 601b, 601c, 601d, etc. which are provided on one or more portions of the outer surface 601. It is understood that the gripping means can be provided in a variety of alternative suitable configurations that provide the desired rotation facilitation. In example embodiments, the outer surface 601 of the adapter 600 and/or one or more other portions of the adapter 600 can also have an amber-color or another color, for example, to indicate syringe contents.

Referring also to FIGS. 6A, 7 and 7A, the cavity 608, which surrounds a periphery of the cavity 604, is dimensioned to receive one or more suitable accessories (e.g., feeding tubes). For example, once the adapter 600 is permanently attached to the syringe 700, as shown in FIGS. 8-8B, an accessory 800, e.g., tubing, having a connector (e.g., a female connector) with dimensions compatible with a connector (e.g., a male connector) formed in cavity 608 may be received in the cavity 608 and removably coupled to the connector in cavity 608. The accessory 800 may also include another connector 830 (e.g., a male connector) that may be coupled to a connector 910 (e.g., a female connector) of a further accessory 900, such as a dispenser for providing fluid from the syringe to a patient. The accessory 800 may, for example, be configured to receive fluid from the syringe 700 via cavity 604 of the adapter 600 and pass the fluid to accessory 900 through a tube 810. The accessory 900 may, in turn, pass the fluid through a tube 920 (e.g., for enteral feeding applications). In example embodiments, each of the above-described connectors is a conventional male or female connector.

The adapter 600 may form a converted or adapted output of the syringe 700 and a rate of fluid flow through the adapter 600 may be based upon dimensions of the adapter 600. It is to be appreciated, however, that the adapter 600 does not change a volume of the syringe 700 when coupled to the syringe 700. It follows that the adapter 600 does not impact accuracy of measurement indicators 714a, 714b, 714c, 714c, 714d on the fluid reservoir 710 that show content levels of fluid in the fluid reservoir 710. Therefore, fluid can be accurately dispensed from the syringe 700 and the adapter 600 to the accessory (or another output).

In example embodiments, the syringe adapter 600 allows for the syringe 700 to be coupled to accessories to which the syringe 700 may not have otherwise been coupled to due to the adapter 600 having a connector which is compatible with an accessory that was previously not compatible with the syringe tip 712. Additionally, due to the above, the syringe adapter 600 may allow for the syringe 700 to be used in applications other than those which the syringe 700 is accustomed to being used. As discussed above, the syringe tip 712 is capable of being coupled to one or more accessories (e.g., feeding tubes) which are compatible with the syringe 700 and the syringe tip 712. With adapter 600, there may also be no need (or a reduced need) to use cleaning brushes for cleaning the adapter 600. Additionally, with adapter 600 permanently attached to syringe 700, an ISO 80369-3 compliant enteral feeding system may be provided. The adapter 600 may also be coupled to ISO 80369-3 compliant enteral feeding tubes (or other feeding tubes depending on the application).

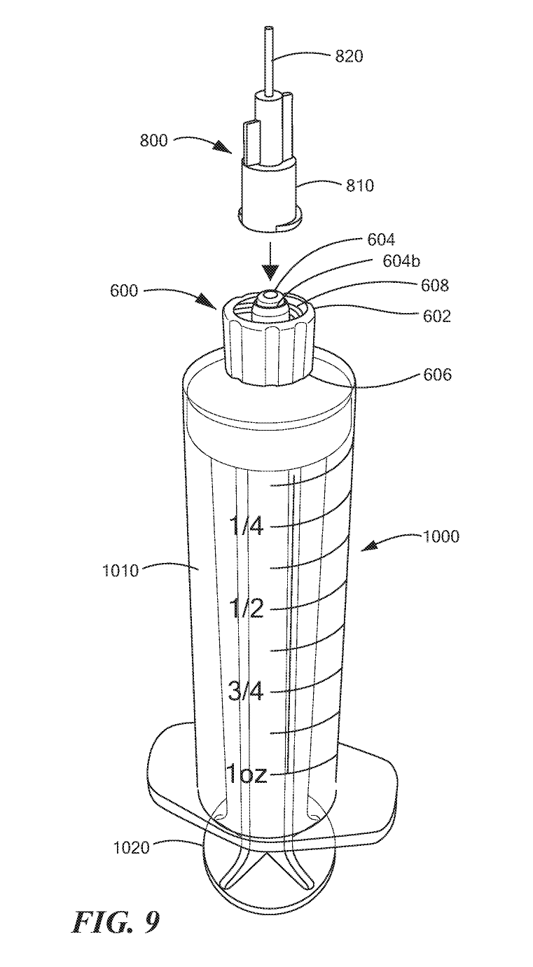

Referring now to FIG. 9, syringe adapter 600 is shown as attached to a syringe 1000. Syringe 1000, similar to syringe 700, can be a conventional enteral or non-luer syringe having a fluid reservoir 1010 from which fluid is forced out by depressing a plunger 1020. Adapter 600 is permanently attached to a syringe tip of the syringe 1000 which is in communication with the fluid reservoir 710. Additionally, adapter 600 may be coupled to accessory 800. In embodiments, the fluid reservoir 1010 and the plunger 1020 of syringe 1000 have one or more dimensions which are larger than corresponding dimensions of reservoir 710 and the plunger 720 of syringe 700.

While the adapter 600 and portions thereof are shown having a particular shapes in FIGS. 6-9, it is understood that the adapter 600 may be of substantially any suitable size and shape for interacting with a syringe tip (e.g., syringe tip 712), which may come in a variety of shapes and forms. In general, the adapter 600 has dimensions sized to meet the needs of a particular application. In the example embodiments described herein, for example, the adapter 600 is shown and described for attachment to a conventional enteral or non-luer syringe (e.g., syringe 700 or syringe 1000). FIGS. 6D and 6E shows certain example dimensions, in inches, for the adapter 600 in accordance with example embodiments described herein. It is understood that while the described drawings may not be to scale, the drawings convey a general dimensional relationship between the components.

Further, while adapter 600 is described as having a cavity 608 with a particular type of connector, it should be appreciated that the type of connector may vary and be selected based on the needs of a particular application. For example, the connector of adapter 600 may be a conventional male connector, a conventional female connector or another type of connector. The connector may comply with the ISO 80369-3 standard (or other applicable standards).

Having described preferred embodiments, which serve to illustrate various concepts, structures and techniques, which are the subject of this patent, it will now become apparent to those of ordinary skill in the art that other embodiments incorporating these concepts, structures and techniques may be used. Additionally, elements of different embodiments described herein may be combined to form other embodiments not specifically set forth above.

Accordingly, it is submitted that that scope of the patent should not be limited to the described embodiments but rather should be limited only by the spirit and scope of the following claims.

What is claimed is:

1. A method, comprising:
securing an adapter to a fluid-dispensing end of a syringe such that the adapter is permanently secured to the syringe, which has indicia corresponding to fluid volume in the syringe, wherein the adapter does not modify the fluid volume of the syringe such that accuracy of the indicia is maintained after the adapter is secured to the syringe.

2. The method according to claim 1, wherein the syringe comprises an enteral feeding syringe.

3. The method according to claim 1 wherein the syringe comprises a non-luer type syringe.

4. The method according to claim 1, wherein the adapter comprises a mating mechanism to secure the adapter to the syringe.

5. The method according to claim 1, wherein the mating mechanism comprises self-threading threads.

6. The method according to claim 5, wherein the mating mechanism is configured for unidirectional rotation.

7. The method according to claim 1, wherein the mating mechanism comprises an adhesive.

8. The method according to claim 1, further including coupling a first accessory to the adapter for receiving fluid from the syringe.

9. The method according to claim 8, wherein the first accessory comprises tubing.

10. The method according to claim 8, further including coupling a second accessory to the first assembly for dispensing fluid to a mouth of a patient.

11. The method according to claim 10, further including providing an ISO-80369-3 compliant enteral feeding system.

12. An assembly, comprising:
a syringe having a fluid reservoir and a tip that is in communication with the fluid reservoir; and
an adapter having first and second ends, the first end including a cavity dimensioned to receive the syringe tip, and a mating mechanism to permanently engage the syringe tip when the syringe tip is received in the cavity, wherein the syringe includes indicia corresponding to fluid volume in the syringe, wherein the adapter does not modify the fluid volume of the syringe such that accuracy of the indicia is maintained after the adapter is secured to the syringe.

13. The assembly according to claim 12, wherein the syringe comprises an enteral feeding syringe.

14. The assembly according to claim 12, wherein the mating mechanism comprises self-threading threads.

15. The assembly according to claim 14, wherein the mating mechanism is configured for unidirectional rotation.

16. The assembly according to claim 12, further including a first accessory coupled to the adapter for receiving fluid from the syringe.

17. The assembly according to claim 16, wherein the first accessory comprises tubing.

18. The assembly according to claim 16, further including a second accessory coupled to the first assembly for dispensing fluid to a mouth of a patient.

19. The assembly according to claim 18, wherein the assembly comprises an ISO-80369-3 compliant enteral feeding system.
